(19) United States
(12) Patent Application Publication
(10) Pub. No.: US 2025/0194913 A1
(43) Pub. Date: Jun. 19, 2025

(54) INTRAORAL SCANNER AND METHOD FOR OBTAINING IMAGE DATA THEREFROM

(71) Applicants: VATECH Co., Ltd., Gyeonggi-do (KR); VATECH EWOO Holdings Co., Ltd., Gyeonggi-do (KR)

(72) Inventors: Jeong Kun KIM, Gyeonggi-do (KR); Tae Han KIM, Gyeonggi-do (KR); Jin Kook ROH, Gyeonggi-do (KR)

(73) Assignees: VATECH Co., Ltd., Gyeonggi-do (KR); VATECH EWOO Holdings Co., Ltd., Gyeonggi-do (KR)

(21) Appl. No.: 18/840,709

(22) PCT Filed: Dec. 20, 2022
(86) PCT No.: PCT/KR2022/020820

§ 371 Date: Aug. 22, 2024

(30) Foreign Application Priority Data
Feb. 23, 2022 (KR) 10-2022-0023713
Dec. 19, 2022 (KR) 10-2022-0177816

(51) Int. Cl. A61B 1/24; A61B 1/00; A61B 1/05; A61B 1/06

(52) U.S. Cl. CPC A61B 1/24; A61B 1/00009; A61B 1/00194; A61B 1/05; A61B 1/0638; A61B 1/0655; A61B 1/0661

(57) ABSTRACT

Disclosed are an intraoral scanner and a method for obtaining image data therefrom. According to the intraoral scanner and method for obtaining image data therefrom according to the present invention, intraoral scanning can be performed using a mono sensor instead of a color sensor and R/G/B color light instead of white light.

### TECHNICAL FIELD

The present disclosure relates to an intraoral scanner and a method for obtaining image data therefrom and, more particularly, to an intraoral scanner and a method for obtaining image data therefrom, which can acquire 3D color image data with high axial and lateral resolutions for a scan object.

### BACKGROUND ART

In general, an intraoral scanner is a device used to obtain three-dimensional image data of teeth and surrounding tissues in the oral cavity, such as the technology disclosed in Korean Patent Application Publication No. 10-2020-0064922. A stereo-type intraoral scanner emits light to an object to be photographed within the oral cavity and receives the light reflected from the object to be photographed by multiple sensors at different positions, and collects three-dimensional (3D) image data of the object using parallax in the images according to sensor positions.

Due to the nature thereof, intraoral scanners are limited in size and have limited axial resolution. For example, when sensors are placed left and right, the farther apart the sensors are, the easier it is to measure the difference in depth of an object being photographed. However, due to the nature of handheld equipment, there are limitations in expanding the size of the equipment left and right, which limits axial resolution.

Meanwhile, conventional intraoral scanners use white light and color sensors to acquire three-dimensional color image data of an object being photographed. Color sensors are disadvantageous in terms of lateral resolution because they need to allocate subpixels for each color within a unit pixel. To increase resolution, structured light with short changeover time is sometimes used, but this complicates the structure of an intraoral scanner and may cause problems such as power consumption and heat generation.

### DOCUMENT OF RELATED ART

### Patent Document

(Patent Document 1) Korean Patent Application Publication No. 10-2020-0064922

### DISCLOSURE

### Technical Problem

The present disclosure is intended to solve the above problems occurring in the related art. An objective of the present disclosure is to provide an intraoral scanner and a method for obtaining image data therefrom, which can acquire 3D color image data with high axial and lateral resolutions for a scan object even with a single sensor.

### Technical Solution

In order to achieve the above mentioned objectives, there is provided an intraoral scanner including: a light source part configured to generate emitted light; a sensor configured to generate image data by receiving reflected light from an object; a controller configured to control the light source part and the sensor; a micro-lens array disposed between the object and the sensor to allow the reflected light to pass through, and configured to include a plurality of micro lenses arranged along a lens surface facing a light-receiving surface of the sensor; and an image processing part configured to generate three-dimensional (3D) image data of the object using the image data.

In addition, there is provided a method for obtaining image data from an intraoral scanner consisting of a light source part, a sensor, a micro-lens array, a controller, and an image processing part, the method including: (a) generating, by the light source part, emitted light so that the emitted light is emitted to an object, and receiving, by the sensor, reflected light that is reflected from the object and transmitted through the micro-lens array; (b) generating, by the sensor, pieces of image data of different parallax levels for the object; and (c) generating, by the image processing part, 3D image data of the object using the pieces of image data of different parallax levels.

### Advantageous Effects

According to an intraoral scanner and a method for obtaining image data therefrom according to an embodiment of the present disclosure, by using a micro-lens array, it is possible to acquire 3D image data with high axial resolution of an object being photographed even with a single sensor.

In addition, according to an intraoral scanner and a method for obtaining image data therefrom according to an embodiment of the present disclosure, by using emitted light of different wavelengths and a mono sensor, it is possible to acquire 3D color image data with high lateral resolution of an object being photographed.

Further scope of applicability of the present disclosure will become apparent from the detailed description that follows. Since various changes and modifications within the spirit and scope of the present disclosure will be readily apparent to those skilled in the art, the detailed description and specific examples, such as preferred embodiments of the present disclosure, should be understood as being given by way of example only.

### DESCRIPTION OF DRAWINGS

FIG. 1 is a schematic view showing the configuration of an intraoral scanner according to an embodiment of the present disclosure;

FIG. 2 is an exemplary block configuration of an intraoral scanner according to an embodiment of the present disclosure;

FIG. 3 is a flowchart of a method for obtaining image data from an intraoral scanner according to an embodiment of the present disclosure;

FIG. 4 is a view showing an example of obtaining a color image using images of different wavelengths; and

FIG. 5 is a schematic view showing the configuration of an intraoral scanner according to another embodiment of the present disclosure.

### BEST MODE

Embodiments disclosed in the present specification will be described in detail with reference to the attached drawings, but identical or similar components will be assigned the same reference numerals regardless of the numbers used in the drawings to identify the components, and redundant descriptions thereof will be omitted. The terms “module” and “part” for components used in the following description are given or used interchangeably only for the ease of preparing the specification, and do not have distinct meanings or roles in themselves. In addition, in describing the embodiments disclosed in this specification, if it is determined that detailed descriptions of related known technologies may obscure the gist of the embodiments disclosed in this specification, the detailed description will be omitted. In addition, the attached drawings are only for easy understanding of the embodiments disclosed in this specification, and the technical idea disclosed in this specification is not limited by the attached drawings. The present disclosure is not limited to the embodiments, but should be understood to include all changes, equivalents, and substitutes included in the spirit and technical scope of the present disclosure.

Terms containing ordinal numbers, such as first, second, etc., may be used to describe various components, but the components are not limited by the terms. The above terms are used solely for the purpose of distinguishing one component from another.

When a component is said to be “connected” or “coupled” to another component, it should be understood that the component may be directly connected to or coupled to the other component, but that other components may exist in between. On the other hand, when a component is said to be “directly connected” or “directly coupled” to another component, it should be understood that there are no other components in between. Singular expressions include plural expressions unless the context clearly dictates otherwise.

It should be further understood that the terms “comprise”, “include”, “have”, etc., when used in this specification, specify the presence of stated features, integers, steps, operations, elements, components, and/or combinations thereof but do not preclude the presence or addition of one or more other features, integers, steps, operations, elements, components, and/or combinations thereof.

In the drawings, the sizes of components may be exaggerated or reduced for convenience of explanation. For example, the size and thickness of each component shown in the drawings are arbitrarily shown for convenience of explanation, and thus the present disclosure is not necessarily limited to what is shown.

In cases where an embodiment may be implemented differently, a specific process sequence may be performed differently from the described sequence. For example, the two processes described in succession may be performed substantially at the same time, or may be performed in the reverse order from the order in which they are described.

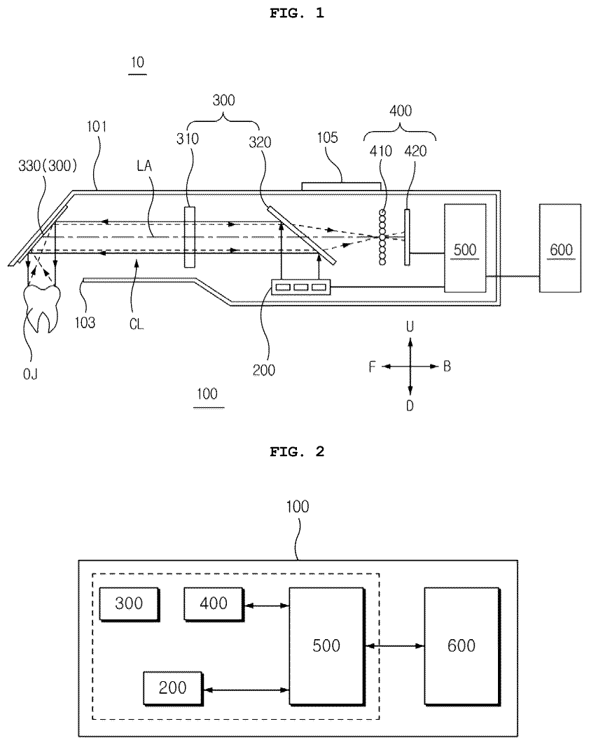

In the following description, one side, the other side, the front side, and the back side may be in the U, D, F, and B directions, respectively, based on an intraoral scanner 100 shown in FIG. 1. In addition, one side may be an upper side and the other side may be a lower side. The up and down direction may be a one side direction.

FIG. 1 is a schematic view showing the configuration of the intraoral scanner 100 according to an embodiment of the present disclosure. FIG. 1 shows an example of the direction in which emitted light and reflected light travel, and the reference numeral for emitted light and reflected light is “CL”.

Referring to FIG. 1, the intraoral scanner 100 according to an embodiment of the present disclosure may include a casing 101 that forms an exterior of the scanner 100.

Inside the casing 101, a light source part 200, an optical part 300, a sensor part 400, and a controller 500 may be accommodated. The casing 101 may have a front-to-back length greater than a vertical width or a left-right width. An opening 103 may be formed on the front lower side of the casing 101. The opening 103 may be a passage through which emitted light from the light source part 200 exits the casing 101 or through which reflected light reflected from a scan object OJ enters the casing 101.

The casing 101 may be provided with an operation part 105. The operation part 105 is for a user to control the operation of the intraoral scanner 100. When the operation part 105 provided in the casing 101 is operated by the user, the intraoral scanner 100 is controlled by the controller 500 to obtain three-dimensional color image data of the object. The operation part 105 may be provided in the casing 101 in the form of hardware. For example, the operation part 105 may be provided in the form of a sensing device, a button, a keypad, a touchpad, etc.

The intraoral scanner 100 according to an embodiment of the present disclosure may include the light source part 200.

The light source part 200 may emit illumination beam, that is, a beam of light. The light source part 200 may emit light of different wavelengths. Different wavelengths may be the red wavelengths, green wavelengths, and blue wavelengths within the visible spectrum. Since they are within the visible spectrum, these wavelengths may refer to color. Hereinafter, color may be used as a term referring to a wavelength range. To be specific, the light source part 200 may emit red light, green light, and blue light individually. To this end, the light source part 200 may include multiple light sources that respectively emit red light, green light, and blue light by single or combined driving. The light sources may be LEDs. The light source part 200 may sequentially emit red light, green light, and blue light for respective frames.

The light source part 200 may be located on the other side of the intraoral scanner 100. Based on FIG. 1, the light source part 200 may be located on the inner lower side of the intraoral scanner 100.

The intraoral scanner 100 according to an embodiment of the present disclosure may include the optical part 300.

The light source part 200 may be arranged along an arbitrary first axis, and the optical part 300 may be arranged along a second axis different from the first axis. The optical part 300 may be located above the light source part 200.

The optical part 300 may include a first reflection part 330.

The first reflection part 330 may be a reflector. The first reflection part 330 may be located on the front side of the casing 101. The first reflection part 330 may be located above the opening 103. The first reflection part 330 may be disposed at an angle so that the lower end thereof is relatively at the front and the upper end thereof is relatively at the rear. As shown in FIG. 1, when the intraoral scanner 100 is positioned near the object OJ for imaging, the opening 103 may be located between the first reflection part 330 and the object OJ. The first reflection part 330 may direct the emitted light produced from the light source part 200 toward the object OJ and direct the reflected light reflected from the object OJ toward the optical part 300.

The optical part 300 may include a beam splitting part 320.

The beam splitting part 320 may direct the emitted light that is emitted along the first axis from the light source part 200 toward the first reflection part 330 arranged on the second axis. The beam splitting part 320 may direct the reflected light that is reflected along the second axis from the reflector 330 toward the sensor part 400, which will be described later.

The beam splitting part 320 may be located above the light source part 200 with reference to FIG. 1. The beam splitting part 320 may include a beam splitter.

The optical part 300 may include a first lens part 310.

The first lens part 310 may include at least one lens.

The first lens part 310 may be positioned between the beam splitting part 320 and the first reflection part 330. The first lens part 310 may be spaced apart from the beam splitting part 320 by a certain distance. The first lens part 310 may be designed in consideration of the working distance, field of view, and optical characteristics of a micro-lens array (MLA) 410, which will be described later.

The intraoral scanner 100 according to an embodiment of the present disclosure may include the sensor part 400.

The sensor part 400 may be arranged coaxially with the optical part 300. That is, the sensor part 400, the beam splitting part 320, the first lens part 310, and the first reflection part 330 may all be arranged coaxially. Thus, the intraoral scanner 100 according to the present disclosure may ensure symmetry. Because of this, the present disclosure may photograph the object OJ under the same conditions even if the intraoral scanner 100 is rotated.

The sensor part 400 may include the micro-lens array 410 and a sensor 420. The sensor 420 and the micro-lens array 410 may detect not only the amount of reflected light but also direction and distance information using a plenoptic or light field method.

The sensor part 400 may be located behind the optical part 300. The sensor 420 may receive reflected light on a frame basis. The sensor 420 may be a mono sensor. The sensor 420 sequentially receives reflected light of different wavelengths reflected from the object OJ by emitted light of different wavelengths sequentially emitted on a frame basis from the light source part 200, thereby generating images in different wavelengths. The sensor 420 may transmit image data in different wavelengths to the controller 500. Different wavelengths may include red wavelengths, green wavelengths, and blue wavelengths.

The sensor part 400 may include the micro-lens array 410. The micro-lens array 410 may be located between the beam splitting part 320 and the sensor 420 of the optical part 300.

The micro-lens array 410 may include a plurality of micro lenses arranged along a lens surface opposing a light-receiving surface of the sensor 420. The micro lenses respectively correspond one-to-one to pixels in the sensor 420, that is, a certain number of pixel groups, and form images of different parallax levels by reflected light. Thus, the sensor may generate image data with different parallax levels for the same object for each pixel group.

The sensor 420 may generate image data with different parallax levels for the object OJ for reflected light of different wavelengths.

The sensor 420 may be an image sensor 420. The sensor 420 may be a complementary metal-oxide-semiconductor (CMOS) image sensor 420. There may be one sensor 420, that is, a single mono sensor 420.

The intraoral scanner of the present disclosure may acquire three-dimensional image data with high axial resolution by means of the micro-lens array 410 and sensor 420.

On the other hand, the micro-lens array 410 composed of the micro lenses may have reduced lateral resolution due to connection points between micro lenses. To overcome this, the light source part 200 of the intraoral scanner of the present disclosure generates emitted light of different wavelengths within the visible spectrum, and the sensor 420 uses a mono sensor that covers the visible spectrum, so that the sensor 420 receives reflected light of different wavelengths to generate image data in different wavelengths. At this time, since the sensor is a mono sensor, the number of effective pixels of the sensor 420 increases compared to a color sensor that requires subpixels for each wavelength band, and thus image data in different wavelengths with high lateral resolution may be generated.

The intraoral scanner of the present disclosure may include the controller 500.

The controller 500 may be connected to the light source part 200. The controller 500 may be connected to the sensor part 400. The controller 500 may control the light source part 200 and the sensor part 400.

The controller 500 may control the light source part 200 and the sensor 420 so that the light source part 200 sequentially emits lights of different wavelengths, and cause the sensor 420 to sequentially receive reflected lights of different wavelengths. To this end, the controller 500 may synchronize (link) the light emission period of the light source part 200 and the light reception period of the sensor 420. In other words, the controller 500 may synchronize the timing at which the light is emitted from the light source part 200 and the timing at which the sensor 420 receives the light. For example, the controller 500 may control the light source part 200 and the sensor 420 to cause the light source part 200 to emit light in red, green, and blue wavelengths, and to cause the sensor 420 to receive reflected light in red, green, and blue wavelengths.

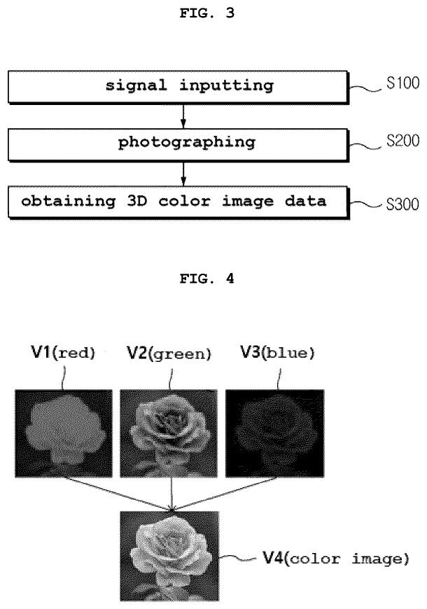

FIG. 2 is a schematic view of the configuration of the intraoral scanner 100.

Referring to FIGS. 1 and 2, the intraoral scanner 100 of the present disclosure may include a processing device 600 (may also be referred to as an image processing part).

The processing device 600 may be connected to the controller 500. The processing device 600 may produce a three-dimensional (3D) color image of the object OJ using pieces of image data of different parallax levels for different wavelengths generated by the sensor 420. The processing device 600 may be configured separately from the casing 101 and connected to the controller 500 wirelessly (Bluetooth, Wi-fi, etc.) or by wire. The processing device 600 and the controller 500 may communicate with each other to transmit and receive information and signals.

The processing device 600 may be configured as a separate computer device. The processing device 600 may be integrated with the casing 101 or may be built into the casing 101.

Meanwhile, the intraoral scanner 100 of the present disclosure may be a handheld type of equipment. Due to the nature of handheld equipment, a time difference may occur between the pieces of image data in different wavelengths received by the sensor, and depending on the time difference, differences may occur in the positions of the object OJ within individual pieces of image data.

Therefore, the processing device 600 may use pieces of image data of different parallax levels for different wavelengths to generate a piece of 3D image data for each of the different wavelengths, determine 3D image data for different wavelengths on the basis of the shape of the object in the pieces of 3D image data for different wavelengths to generate overlapping 3D image data overlaid on some areas of the object, and generating 3D color image data for some areas of the object by reflecting colors of different wavelengths in the overlapping 3D image data. This operation is carried out throughout the entire oral cavity by moving the intraoral scanner, and 3D color image data for some areas of an object may be stitched together to obtain 3D color image data for the entire object.

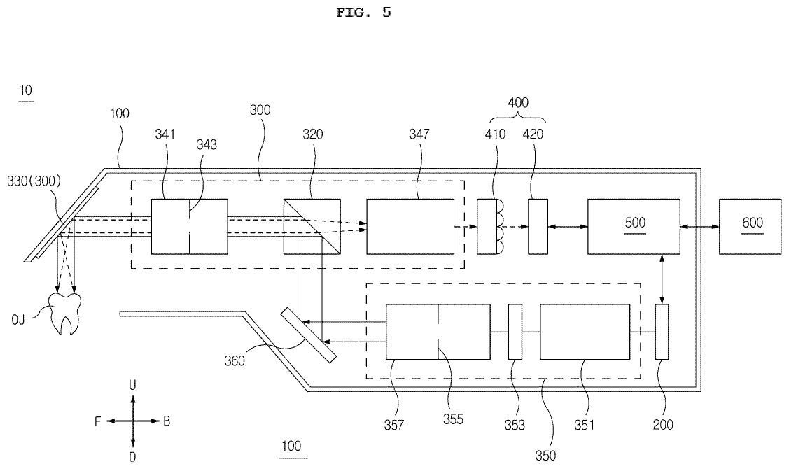

FIG. 3 is a flowchart of a method for obtaining image data from the intraoral scanner 100 according to an embodiment of the present disclosure. FIG. 4 is a view showing an example of obtaining one color image V4 by combining three different wavelength images V1, V2, and V3.

Hereinafter, an example of the method for obtaining image data from the intraoral scanner 100 will be described with reference to FIGS. 1 to 4.

The method for obtaining image data from the intraoral scanner 100 according to an embodiment of the present disclosure may include signal inputting S100, photographing S200, and obtaining 3D color image data S300.

The signal inputting S100 may be a step in which a signal for photographing the object OJ is input to the controller 500 by a user.

Photographing S200 may be a step in which lights of different wavelengths are sequentially emitted from the light source part 200 under the control of the controller 500, and reflected lights of different wavelengths are sequentially received by the sensor 420 under the control of the controller 500 to generate pieces of image data in different wavelengths.

To be specific, the photographing $200 may include: generating image data in a first wavelength range as the light source part 200 emits light of the first wavelength range and the sensor 420 of the sensor part 400 receives reflected light of the first wavelength range under the control of the controller 500; transferring image data in the first wavelength range from the sensor part 400 to the controller 500; transferring image data in the first wavelength range from the controller 500 to the processing device 600;

generating image data in a second wavelength range as the light source part 200 emits light of the second wavelength range and the sensor 420 of the sensor part 400 receives reflected light of the second wavelength range under the control of the controller 500; transferring image data in the second wavelength range from the sensor part 400 to the controller 500; transferring image data in the second wavelength range from the controller 500 to the processing device 600;

generating image data in a third wavelength range as the light source part 200 emits light of the third wavelength range and the sensor 420 of the sensor part 400 receives reflected light of the third wavelength range under the control of the controller 500; transferring image data in the third wavelength range from the sensor part 400 to the controller 500; and transferring image data in the third wavelength range from the controller 500 to the processing device 600. In this case, the first, second, third wavelength ranges may be red, green, and blue light wavelengths, respectively.

Obtaining 3D color image data S300 may be a step in which the processing device 600 acquires three-dimensional color image data for an area of the object OJ using image data in the first to third wavelength ranges.

The obtaining of 3D color image data S300 may include: generating, by the processing device 600, a piece of 3D image data for each wavelength range using image data of different parallax levels included in the image data of the first to third wavelength ranges; matching the pieces of 3D image data based on the shape of the object in the 3D image data for each wavelength range to generate overlapping 3D image data overlaid on an area of the object, where the shapes overlap with each other; and obtaining 3D color image data for the area of the object by reflecting colors for respective wavelength ranges in the overlapping 3D image data.

FIG. 4 shows the principle of generating a color image for an object with first to third images in different wavelength ranges for the same object.

FIG. 5 is a schematic view showing the configuration of an intraoral scanner according to another embodiment of the present disclosure.

When compared to the embodiment of FIG. 1, the biggest difference of this embodiment is that it includes a pattern part 350 that imparts a pattern to emitted light of the light source. Hereinafter, the emitted light with a pattern is referred to as patterned light. Preferably, a first aperture part 357 for adjusting an F value of emitted light or patterned light and a second aperture part 341 for adjusting an F value of reflected light may be included.

To be specific, the intraoral scanner according to another embodiment of the present disclosure includes the pattern part 350 and a second reflection part 360 provided between the light source part 200 and the beam splitting part 320 as shown in FIG. 5.

The pattern part 350 is to convert emitted light from the light source part 200 into patterned light. The pattern part 350 may include at least one lens 351, a pattern filter 353, and the first aperture part 357. The lens 351 may focus the emitted light from the light source part 200 into parallel light. The pattern filter 353 may convert the parallel light into patterned light by imparting a pattern to the parallel light. The first aperture part 357 adjusts the F value of the patterned light. The first aperture part 357 may include at least one lens and a first aperture 355.

The second reflection part 360 may be a reflector.

The second reflection part 360 reflects the patterned light whose F value is adjusted through the first aperture part 357 to the beam splitting part 320.

The intraoral scanner according to another embodiment of the present disclosure may include a second aperture part 341 provided between the beam splitting part 320 and the first reflection part 330, and a second lens part 347 provided between the sensor part 400 and the beam splitting part 320.

The second aperture part 341 adjusts the F value of reflected light. The second aperture part 341 may include at least one lens and a first aperture 343. In this case, at least one lens may serve as the first lens part (see 310 in FIG. 1) of the previous embodiment.

The second lens part 347 includes at least one lens, and processes the reflected light whose F value is adjusted by the second aperture part 341 and transmits the processed reflected light to the sensor part 400.

The diameter of the second aperture 343 is preferably larger than that of the first aperture 355 so as to affect reflected light only, that is, not to affect emitted light or patterned light. In this way, by using the second aperture as an optical path for emitted light or patterned light and reflected light, and controlling only the F value of the reflected light, the product size may be miniaturized by reducing the number of parts.

Meanwhile, in the intraoral scanner according to the present disclosure, to facilitate long-focus or short-focus implementation, the micro-lens array 410 may be a solid immersion micro-lens array in which a solid surface with a different refractive index than the micro lens is immersed along the lens surface.

For reference, when the focal length of the micro lens is

$${f}_{ML} = \frac{R}{{n}_{ML} - 1} ,$$

the focal length (ƒ_{sl-Mt}) of the solid immersion micro-lens array is desirable to have the following mathematical relationship.

$${f}_{SI - ML} = \frac{{n}_{ML} - 1}{{n}_{ML} - {n}_{SL}} {f}_{ML} + ( 1 - \frac{1}{{n}_{SL}} ) {t}_{SL}$$

In the above equation, R is the curvature of the micro lens, n_{ML} is the curvature of the micro-lens array, and n_{SL} is the refractive index of a solid layer.

Furthermore, the solid layer of the solid immersion micro-lens array is preferably made of any one selected from the group consisting of homopolymer, composite polymer, oxide, nitride, and carbide. As an example, the solid layer may be silicon, PDMS, PMMA, PI, SiO₂, SiₓN_{y}, etc.

In addition, the thickness of the solid layer of the solid layer of the solid immersion micro-lens array is preferably 1 μm to 100 μm.

In this specification, image may mean data. The image may include the shape of the scan object OJ. The object OJ may mean at least one of the interior of the oral cavity, a tooth, teeth, and tissues surrounding the teeth. The processing device 600 may be a personal computer, a laptop computer, a tablet PC, a mobile phone, or a smartphone. A beam may be understood as light. Although not shown in the drawings, the processing device 600 may also be provided with an operation part. In this case, the operation part may be provided in the form of software in the processing device 600.

Any or other embodiments of the present disclosure described above are not exclusive or distinct from each other. In certain embodiments or other embodiments of the present disclosure described above, each configuration or function may be used in combination or combined.

It is obvious to those skilled in the art that the present disclosure may be embodied in other specific forms without departing from the spirit and essential features of the present disclosure. The above detailed description should not be construed as limiting in any respect and should be considered illustrative. The scope of the present disclosure should be determined by reasonable interpretation of the appended claims, and all changes within the equivalent scope of the present disclosure are included in the scope of the present disclosure.

### DESCRIPTION OF REFERENCE NUMERALS

100: intraoral scanner 101: casing 103: opening
105: operation part 200: light source part 300: optical part
310: first lens part 320: beam splitting part 330: first reflection part
400: sensor part 410: micro-lens array
420: sensor 500: controller 600: processing device
CL: light OJ: object
LA: light axis V1: first image V2: second image
V3: third image V4: fourth image

1. An intraoral scanner, comprising:
a light source part configured to generate emitted light;
a sensor configured to generate image data by receiving reflected light from an object;
a controller configured to control the light source part and the sensor;
a micro-lens array disposed between the object and the sensor to allow the reflected light to pass through, and configured to include a plurality of micro lenses arranged along a lens surface facing a light-receiving surface of the sensor; and
an image processing part configured to generate three-dimensional (3D) image data of the object using the image data.

2. The intraoral scanner of claim 1, wherein the light source part generates emitted lights of different wavelengths,
the sensor is a mono sensor,
the controller causes the light source part to sequentially generate the emitted lights of different wavelengths and the sensor to sequentially receive reflected lights of different wavelengths to generate image data in different wavelengths, and
the image processing part generates 3D color image data of the object using the image data in different wavelengths.

3. The intraoral scanner of claim 2, wherein the different wavelengths include red wavelengths, green wavelengths, and blue wavelengths within a visible spectrum.

4. The intraoral scanner of claim 1, further comprising:
a first aperture provided between the light source part and the object to adjust an F value of the emitted light.

5. The intraoral scanner of claim 1, further comprising:
a second aperture provided between the object and the micro-lens array to adjust an F value of the reflected light.

6. The intraoral scanner of claim 1, further comprising:
a pattern part provided between the light source part and the object to impart a pattern to the emitted light.

7. The intraoral scanner of claim 1, further comprising:
a first aperture provided between the light source part and the object to adjust an F value of the emitted light; and
a second aperture provided between the object and the micro-lens array to adjust an F value of the reflected light,
wherein the light source part and the first aperture are arranged along a first axis, whereas the second aperture and the sensor are arranged along a second axis different from the first axis,
a diameter of the first aperture is smaller than a diameter of the second aperture, and
a beam splitter is further provided between the first aperture and the second aperture to direct the emitted light passing through the first aperture toward the object through the second aperture and to direct the reflected light passing through the second aperture toward the micro-lens array.

8. The intraoral scanner of claim 1, wherein the micro-lens array is a solid immersion micro-lens array in which a solid layer with a different refractive index than the plurality of micro lenses is immersed along the lens surface.

9. A method for obtaining image data from an intraoral scanner consisting of a light source part, a sensor, a micro-lens array, a controller, and an image processing part, the method comprising:
(a) generating, by the light source part, emitted light so that the emitted light is emitted to an object, and receiving, by the sensor, reflected light that is reflected from the object and transmitted through the micro-lens array;
(b) generating, by the sensor, pieces of image data of different parallax levels for the object; and
(c) generating, by the image processing part, 3D image data of the object using the pieces of image data of different parallax levels.

10. The method of claim 9, wherein the light source part generates emitted lights of different wavelengths,
the sensor is a mono sensor,
the controller causes, in the step (a), the light source part to sequentially generate the emitted lights of different wavelengths and the sensor to sequentially receive reflected lights of different wavelengths,
the sensor generates, in the step (b), the pieces of image data of different parallax levels for the different wavelengths, and
the image processing part generates, in the step (c), the 3D image data of the object using the pieces of image data of different parallax levels generated for the individual different wavelengths.
